(19) United States
(12) Patent Application Publication
(10) Pub. No.: US 2023/0044684 A1
(43) Pub. Date: Feb. 9, 2023

(54) RAPID PRECIPITATION-DRIVEN KILOBASE SIZE SELECTION OF HMW DNA

(71) Applicant: PACIFIC BIOSCIENCES OF CALIFORNIA, INC., Menlo Park, CA (US)

(72) Inventors: Kelvin Jeng-Fang LIU, Baltimore, MD (US); John Duncan KILBURN, Baltimore, MD (US)

(21) Appl. No.: 17/784,474

(22) PCT Filed: Dec. 11, 2020
(86) PCT No.: PCT/US2020/064508

§ 371 Date: Jun. 10, 2022

Related U.S. Application Data
(60) Provisional application No. 62/947,969, filed on Dec. 13, 2019.

(51) Int. Cl. C12N 15/10

(52) U.S. Cl. CPC C12N 15/1003

(57) ABSTRACT

Provided herein are methods of purifying a sample containing nucleic acids to obtain isolated nucleic acids of a desired size range and methods of sequencing nucleic acids of a desired size range. The methods include a) combining a nucleic acid-containing sample with a precipitation buffer in a container to provide a precipitation mixture in which the precipitation buffer comprises water, a buffer, a salt, and polyvinyl pyrrolidinone (PVP) and/or Ficoll. The methods also include precipitating the nucleic acids in the precipitation mixture to provide a precipitated nucleic acid portion and a remaining sample portion. The precipitated nucleic acid portion predominantly comprises nucleic acid molecules above a selected size cutoff value and the remaining sample portion predominantly comprises nucleic acid molecules below the selected size cutoff value. The methods also include separating the precipitated nucleic acid portion from the remaining sample portion. Related compositions and kits are also provided herein.

### CROSS-REFERENCE TO RELATED APPLICATIONS

This application claims the benefit of, and relies on the filing date of, U.S. provisional patent application No. 62/947,696, filed 13 Dec. 2019, the entire disclosure of which is incorporated herein by reference.

### BACKGROUND

In recent years, 3rd generation sequencing technologies have revolutionized our understanding of the structure-function of the genome and the accuracy of reference assemblies. Transformative advances from Pacific Biosciences (Menlo Park, Calif.), Oxford Nanopore Technologies Limited (Oxford, United Kingdom), 10× Genomics (Pleasanton, Calif.) and Bionano Genomics (San Diego, Calif.) have created a resurgent need for high molecular weight (HMW) DNA of the utmost quality and for new technologies to effectively process it. However, the vast majority of technologies to process and analyze HMW DNA, such as pulsed field gel electrophoresis (PFGE), gel plug extractions and dialysis purification were originally developed in the infancy of molecular biology and are incredibly slow and tedious.

Library preparation for most long-read sequencing technologies follows a similar workflow. First, HMW DNA (50 kb-Mb+) must be isolated. Next, the DNA is prepared for sequencing using various enzymatic steps. During enzymatic processing, size selection is used to remove smaller background molecules from the desired library products. This is done almost exclusively with Beckman Coulter AMPURE® beads. The size selection cutoffs for AMPURE® (100 bp-1000 bp) are too low for most long-read libraries. Thus, a follow-up size selection is often performed using a PFGE instrument such as Sage Science's BLUEPIPPIN™ (Beverly, Mass.) to enhance read lengths by isolating only the highest molecular weight library products. While BLUEPIPPIN™ can size select large DNA (100 bp-50 kb), it is slow (8.5 hours) and also damages DNA during the long PFGE process, necessitating subsequent enzymatic repair. All size selection steps must work optimally at relatively high concentrations (>50 ng/μl); as sequencing lengths desired increase, the mass concentration of DNA must also increase in order to keep a constant molarity. Thus, for reads in the range 100 kbp to 1 Mbp the mass concentration needs to be 200-3000 times higher than samples with the same molarity that have fragment lengths of 350-600 bp typical of Illumina sequencing. The recoveries of both AMPURE® and BLUEPIPPIN™ are impaired at high concentrations.

Accordingly, there remains a desire in the art for technologies capable of rapid size selection of large nucleic acid molecules in the size range >10 kb that does not require separate AMPURE® and PFGE purification steps and which does not damage the nucleic acids during processing.

### SUMMARY

In one aspect, the present disclosure provides a method of purifying a sample containing nucleic acids to obtain isolated nucleic acids of a desired size range. The method includes a) combining a nucleic acid-containing sample with a precipitation buffer in a container to provide a precipitation mixture, wherein the precipitation buffer comprises water, a buffer, a salt, and polyvinylpyrrolidone (PVP) and/or Ficoll. The method also includes b) precipitating the nucleic acids to provide a precipitated nucleic portion and a remaining sample portion, wherein precipitated portion predominantly comprises nucleic acid molecules above a selected size cutoff value and wherein the remaining sample portion predominantly comprises nucleic acid molecules below the selected size cutoff value. In addition, the method also includes c) separating the precipitated nucleic acid from the remaining sample portion, thereby obtaining the isolated nucleic acids of the desired size range.

In one aspect, the present disclosure provides a method of sequencing nucleic acids of a desired size range. The method includes a) combining a nucleic acid-containing sample with a precipitation buffer in a container to provide a precipitation mixture, wherein the precipitation buffer comprises water, a buffer, a salt, and polyvinylpyrrolidone (PVP) and/or Ficoll. The method also includes b) pelleting the nucleic acids in the precipitation mixture to provide a nucleic acid pellet and a remaining sample portion, wherein the nucleic acid pellet predominantly comprises nucleic acid molecules above a selected size cutoff value and wherein the remaining sample portion predominantly comprises nucleic acid molecules below the selected size cutoff value. The method also includes c) separating the nucleic acid pellet from the remaining sample portion to produce isolated nucleic acids of the desired size range. In addition, the method also includes d) sequencing the isolated nucleic acids of the desired size range to produce sequencing reads, thereby sequencing the nucleic acids of the desired size range.

In one aspect, the present disclosure provides a method of sequencing nucleic acids of a desired size range. The method includes a) combining a nucleic acid-containing sample with a precipitation buffer in a container to provide a precipitation mixture, wherein the precipitation buffer comprises water, a buffer, a salt, and polyvinylpyrrolidone (PVP) and/or Ficoll. The method also includes b) precipitating the nucleic acids in the precipitation mixture to provide a precipitated nucleic acid portion and a remaining sample portion, wherein the precipitated nucleic acid portion predominantly comprises nucleic acid molecules above a selected size cutoff value and wherein the remaining sample portion predominantly comprises nucleic acid molecules below the selected size cutoff value. The method also includes c) separating the nucleic acid pellet from the remaining sample portion to produce isolated nucleic acids of the desired size range. In addition, the method also includes performing steps a)-c) at any step during a sequencing library preparation, for example after an end-prep/dA tailing reaction or an adapter ligation reaction.

In some embodiments, the PVP comprises a molecular weight (MW) selected from the group consisting of: MW10,000, MW29,000, MW40,000, MW55,000, MW360,000, MW1,300,000, or other molecular weights between MW5,000 and MW5,000,000. In certain embodiments, a concentration of the PVP in the precipitation buffer is 0.01%-40% weight/volume (w/v %). In some embodiments, the Ficoll comprises a molecular weight (MW) selected from the group consisting of: 70,000, 400,000, or other molecular weights between MW5,000 and MW5,000,000. In certain embodiments, a concentration of the Ficoll in the precipitation buffer is 0.01%-60% weight/volume (w/v %). In some embodiments, the selected size cutoff value is from 50 bp-1,000 kilobases (kb). In some embodiments, nucleic acid molecules in the nucleic acid-containing sample comprise a concentration range of between about 1-2000 ng/μL. In some embodiments, the salt comprises one or more of: guanidinium chloride, guanidinium hydrochloride, lithium perchlorate, guanidinium thiocyanate, guanidinium isothiocyanate, sodium chloride, potassium chloride, magnesium chloride, calcium chloride, lithium chloride, sodium acetate, potassium acetate, and sodium iodide.

In certain embodiments, the methods disclosed herein include tuning at least one condition of the precipitation buffer to determine the selected size cutoff value. In these embodiments, the condition is typically selected from the group consisting of: PVP concentration, PVP molecular weight, Ficoll concentration, Ficoll molecular weight, presence or absence of chaotropic salts, presence or absence of monovalent and/or divalent salts, salt concentration and type, alcohol type and concentration, presence or absence of polyamines, presence or absence of denaturing agents, presence or absence of other additive molecules, pH, precipitation/binding time, precipitation/binding temperature, precipitation/binding volume, centrifugation time, centrifugation temperature, and combinations thereof.

In some embodiments, step b) in [0005] comprises centrifuging the precipitation mixture. Typically, step b) comprises centrifuging the precipitation mixture at 10000 g for 30 minutes at room temperature (RT). In certain embodiments, the remaining sample portion comprises supernatant and wherein step c) comprises removing the supernatant from the container. In some embodiments, the methods further include a) washing the nucleic acid pellet one or more times with an alcohol solution to produce a washed nucleic acid pellet, and b) resuspending the washed nucleic acid pellet in a resuspension buffer to produce resuspended nucleic acids. In some of these embodiments, the resuspension buffer comprises a TE buffer or a low EDTA TE buffer.

In some embodiments, the method further includes prior to step a) in [0005] combining the nucleic acid-containing sample with a binding buffer to provide a binding mixture; contacting the binding mixture with a nanomembrane, wherein the nanomembrane binds nucleic acids in the binding mixture to produce bound nucleic acids; and separating the bound nucleic acids from remaining components of the binding mixture. In certain embodiments, the method further includes contacting the nucleic acids with a nanomembrane in the precipitation mixture, wherein the nanomembrane binds nucleic acids in the precipitation mixture to produce bound nucleic acids. In some embodiments, the method further includes during or after step c) in [0005] contacting the resuspended nucleic acids with a nanomembrane, wherein the nanomembrane binds nucleic acids in the resuspension buffer to produce bound nucleic acids; and separating the bound nucleic acids from remaining components of the resuspension buffer.

In some embodiments, the methods further include sequencing the isolated nucleic acids of the desired size range after step c) in [0005] to produce sequencing reads. Typically, an N50 of the sequencing reads obtained after performing at least steps a)-c) is increased relative to an N50 of sequencing reads obtained in the absence of performing steps a)-c).

In one aspect, the present disclosure provides a method of removing RNA from a total nucleic acid sample. In this aspect the input nucleic acid for steps a)-c) in [0005] is extracted from biological samples without RNase treatment and so includes both DNA and RNA. The size distributions of extracted DNA and RNA are significantly different, with the RNA distribution being on average smaller than the DNA. Thus, the size selection in step [0005] also selects for RNA or DNA. The pellet in step b) of [0005] will contain nucleic acid with enriched DNA content and the remaining sample portion contains nucleic acid with enriched RNA content. Separating the nucleic acid pellet from the remaining sample portion in step c) of [0005], thereby obtains a DNA enriched fraction and an RNA enriched fraction for use in further analyses.

In another aspect, separation of DNA and RNA may be performed by exploiting solubility differences between double stranded and single stranded nucleic acids in addition to size differences.

In one aspect, the present disclosure provides a method of removing impurities from nucleic acid samples, while retaining intact DNA. Soluble impurities do not precipitate in the method described in [0005], thus when the remaining sample is removed from the precipitated nucleic acids those impurities are also removed. This can be observed in, for example, 260/230 ratios in UV spectroscopy approaching those expected of pure DNA after size selection.

In another aspect, the present disclosure provides a composition that includes nucleic acids and a precipitation buffer. The precipitation buffer comprises water, a buffer, a salt, and polyvinylpyrrolidone (PVP) and/or Ficoll. A portion of the nucleic acids are present in a nucleic acid pellet that predominantly comprises nucleic acid molecules above a selected size cutoff value and a remaining portion of the nucleic acids are present in a supernatant that predominantly comprises nucleic acid molecules below the selected size cutoff value. In some embodiments, the PVP comprises a molecular weight (MW) selected from the group consisting of: MW10,000, MW29,000, MW40,000, MW55,000, MW360,000, and MW1,300,000 or other molecular weights between MW5,000 and MW5,000,000. In certain embodiments, a concentration of the PVP in the precipitation buffer is 0.1%-40% weight/volume (w/v %). In some embodiments, the Ficoll comprises a molecular weight (MW) selected from the group consisting of: MW70,000, and MW400,000, or other molecular weights between MW5,000 and MW5,000,000. In certain embodiments, a concentration of the Ficoll in the precipitation buffer is 0.01%-40% weight/volume (w/v %). In some embodiments, the selected size cutoff value is from 1-100 kilobases (kb).

In another aspect, the present disclosure provides a kit for purifying a sample containing nucleic acids to obtain isolated nucleic acids of a desired size range. The kit includes a buffer, a salt, and polyvinylpyrrolidone (PVP) and/or Ficoll disposed in one or more containers. In some embodiments, the kit includes a single container that comprises a precipitation buffer that comprises water, the buffer, the salt, and the PVP and/or Ficoll. In certain embodiments, the kit further includes a nanomembrane. In some embodiments, the kit further includes one or more wash buffers and/or one or more elution buffers. In certain embodiments, the kit further includes one or more sequencing reagents.

In another aspect, the present disclosure provides for a range of kits for purifying a sample containing nucleic acids to obtain isolated nucleic acids of a desired size range with different kits having different size ranges. The kits include: 1) a “Short Read Eliminator XS” kit, with near complete removal of DNA below 5 kilobases and progressive depletion up to 10 kilobases; 2) a “Short Read Eliminator” kit, with near complete removal of DNA below 10 kilobases and progressive depletion up to 25 kilobases; 3) a “Short Read Eliminator XL” kit, with near complete removal of DNA below 10 kilobases and progressive depletion up to 40 kilobases.

### BRIEF DESCRIPTION OF THE FIGURES

FIG. 1A depicts size selection purification of λ DNA spiked with a 1 kb plus ladder as described in Example 1 using a size select method as described herein. The nucleic acid size select method was performed using polyvinylpyrrolidone (PVP)-driven precipitation. PVP acts as a molecular crowder to tune nucleic acid precipitation by length. FIG. 1B depicts the cutoff size (defined as the highest Mw band that has 10% or lower recovery) that was tunable from 1000 bp-10 kb by changing PVP concentration. See Example 1.

FIG. 2 depicts an exemplary protocol for the size selection of high molecular weight (HMW) genomic DNA (gDNA).

FIG. 3A depicts an image of a 1% agarose gel separation of HMW gDNA size-selected using the Short Read Eliminator (SRE) Kit as described in Example 2. Sizing cutoffs were demonstrated using a spiked-in ladder (Thermo Scientific GeneRuler 1 kb Plus, #SM1334). Input was 50 ng/μL gDNA extracted from GM12878 cells using the NANOBIND™ CBB Big DNA Kit+20 ng/μL ladder.

FIG. 3B depicts an image of the gel separation of the sample from FIG. 3A analyzed using an Agilent TapeStation 4200.

FIG. 4 is a graph showing results of Oxford Nanopore MinION/GridION sequencing in which DNA samples were prepared using the NANOBIND™ CBB Big DNA Kit alone or the NANOBIND™ CBB Big DNA Kit in combination with the Short Read Eliminator (SRE) Kit as described in Example 4. The x-axis shows read length in kilobases (kb), while the y-axis shows the normalized data.

FIG. 5 is a graph showing results of Oxford Nanopore PromethION sequencing in which DNA samples were prepared using a Qiagen Puregene kit alone or the Qiagen Puregene kit in combination with the Short Read Eliminator (SRE) Kit as described in Example 5. The x-axis shows read length in kilobases (kb), while the y-axis shows the normalized data.

FIG. 6 is a graph showing results of Oxford Nanopore MinION sequencing in which the Short Read Eliminator (SRE) Kit was used instead of AMPure beads to clean up the reactions in the SQK-LSK109 library preparation as described in Example 6. The x-axis shows read length in kilobases (kb), while the y-axis shows the normalized data.

FIG. 7 is a graph showing % recovery of DNA and RNA fractions of a nucleic acid sample extracted from E. coli cells as described in Example 7. For SRE XS, 94% of the RNA did not pellet and therefore was removed when the precipitation buffer was separated from the pellet after centrifugation, leaving a DNA-enriched fraction.

FIG. 8 depicts size selection purification of gDNA spiked with a 1 kb plus ladder as described in Example 13 using the precipitation method as described in [0005]. The size select method was performed using Ficoll-driven precipitation. Ficoll acts as a molecular crowder to tune nucleic acid precipitation by length. Precipitation in 20% Ficoll and 0.75 M NaCl returns DNA above 1.5-3 kb.

### DETAILED DESCRIPTION

The present disclosure is directed to methods for the rapid size selection of nucleic acid molecules from nucleic acid-containing samples using a precipitation buffer comprising PVP and/or Ficoll, salt and buffer.

Next generation sequencing (NGS) is experiencing tremendous growth driven by both research and clinical applications. 3^{rd} generation sequencing technologies are being used to de novo sequence an ever-growing catalog of plants, animals, and microbes while continually refining the quality of human reference genomes. NGS is also being used to better understand fundamental biology such as genetic diversity, metagenomics, and epigenetics. However, the maturation of clinical tests such as liquid biopsies, non-invasive prenatal testing, and infectious disease testing will likely grow to be the major driving force in the near future.

Size selection purification is an important part of many NGS library preparations. A size selection step is often used to isolate molecules of a specific size before proceeding with the library preparation. Alternatively, a size selection step can also be used to isolate only library molecules of a specific size just prior to sequencing. In long-read NGS, excess short DNA compromises mean read lengths and reduces assembly quality or identification of structural variants.

As described herein, a size selection process using a PVP and/or Ficoll containing precipitation buffer, such as that contained in the Short Read Eliminator (SRE) kit, results in tunable cutoffs of nucleic acids ranging in size between 1000 bp-100 kb. The size select process can be used to eliminate nucleic acid molecules below the cutoff, for example, to obtain sequencing libraries for enhanced sequencing read lengths. As described herein below, specific polymers may be used to separate the undesired short DNA from the desired long DNA to use as input for a library preparation, and to speed overall library preparation by replacing slow and tedious PFGE separation with an instant rapid precipitate, wash, and elute process. Only specific polymers can be used to facilitate high sizing cutoffs, fast processing time, and high recovery. High purification efficiency (e.g., >99%) across, e.g., the 100 bp-2000 kb range can be achieved with high efficiency recovery (e.g. >90%) of high MW DNA (e.g., 50 kb-1 Mb+) rapidly, e.g., <1 hour process.

As also described herein proteins can be co-purified with nucleic acid. The aim of most purification methods is to take a reaction mixture and return only the size selected nucleic acid fraction whilst removing all other reagents, including but not limited to enzymes, buffers and dNTPs. As described herein, methods which allows nucleic acid and proteins to be purified together in some embodiments such that both nucleic acids and enzymes, for example, can be isolated from the rest of the reagents in a co-purification. The size cutoff value describes a threshold below which nucleic acid molecules are inefficiently recovered and above which nucleic acid molecules are efficiently recovered. A cutoff size can be defined as the size of nucleic acid whose recovery is halfway between the limiting behavior of the low size recovery and the high size recovery. For example, if the low molecular weight nucleic acid molecules (e.g. 10 bp) are recovered with 0% efficiency and high molecular weight nucleic acid molecules (e.g. 50 kbp) are recovered with 100% efficiency, then the cut-off size is the molecular weight that has 50% recovery.

The size cutoff value can also describe a threshold above which nucleic acid molecules are inefficiently recovered and below which nucleic acid molecules are efficiently recovered. A cutoff size can be defined as the size of nucleic acid whose recovery is halfway between the limiting behavior of the low size recovery and the high size recovery. For example, if the low molecular weight nucleic acid molecules (e.g. 10 bp) are recovered with 100% efficiency and high molecular weight nucleic acid molecules (e.g. 50 kbp) are recovered with 0% efficiency, then the cut-off size is the molecular weight that has 50% recovery.

As used herein, the term “nucleic acid(s)” or “nucleic acid molecule(s)” are used interchangeably and include “polynucleotide(s)” and “oligonucleotide(s).” The term further includes a polymer of DNA, RNA or cDNA, which can be single-stranded or double stranded, synthesized or obtained (e.g., isolated and/or purified) from natural sources, which can contain natural, non-natural or altered nucleotides, and which can contain a natural, non-natural or altered internucleotide linkage, such as a phosphoramidate linkage or a phosphorothioate linkage, instead of the phosphodiester found between the nucleotides of an unmodified oligonucleotide. The term further includes nucleic acids with other common nucleic acid modifications, including but not limited to fluorophores, quenchers, methylated bases. The nucleic acids to be processed may be described as genomic DNA (gDNA), mitochondrial DNA (mtDNA), plasmid DNA (pDNA), cell-free DNA (cfDNA), circulating nucleic acids, cell-free RNA (cfRNA), microRNA, ribosomal RNA (rRNA), messenger RNA (mRNA), transfer RNA (tRNA), non-coding RNA (ncRNA). High molecular weight DNA is large unfragmented DNA that is typically greater than 20 kb in length, often hundreds of kb in length (up to 100 kb, 200 kb, 300 kb, 500 kb, etc.) and sometimes Mb in length (up to 1 Mb, 2 Mb, 5 Mb+, etc.).

As used herein, the term ‘desired size range’ when used in reference to DNA sizes, is used to describe a set of DNA sizes that are a subset of the DNA sizes contained in the input to a described protocol step, or the size selection process in its entirety. As an example, the input DNA sample is a library preparation product that contains DNA with lengths between 10 bp and 500,000 bp; there are no DNA molecules shorter than 10 bp and none longer than 500,000 bp in the sample. All other sizes are represented with equal number. The desired size range in this example is all DNA molecules above a cutoff size of 10000 bp. Thus, the desired size range comprises the subset of input molecules containing DNA with lengths between 10000 bp and 500,000 bp; there are no DNA molecules in the desired size range shorter than 10000 bp and none longer than 500,000 bp. As another example, the limits are defined in terms of percentage recoveries of DNA greater than or lower than that DNA size. With the same input sample as above: DNA with lengths between 10 bp and 500,000 bp; there are no DNA molecules shorter than 10 bp and none longer than 500,000 bp in the sample. All other sizes are represented with equal number. The desired size range comprises a recovery such that the average recovery for DNA molecules with lengths greater than 10000 bp is 90%, and the average recovery for DNA molecules with lengths shorter than 10000 bp is 10%. Thus, the longer DNA molecules are preferentially recovered.

The nucleic acid-containing sample, such as a DNA-containing sample, comprises nucleic acids, such as DNA molecules, of different sizes (lengths). The method according to the present disclosure allows for the size selection of single stranded as well as of double-stranded nucleic acids. Typically, the nucleic acid molecules are linear, double-stranded DNA molecules. However, they made also be single stranded DNA molecules, single stranded RNA molecules, or double stranded RNA molecules. The nucleic acid-containing sample can be of various origins, including biological samples and artificial samples that are obtained during nucleic acid processing. Biological samples can include body fluids such as blood, plasma, serum, urine, feces, sputum, buccal swabs, hair, teeth, bone or other clinical samples such as cultured cells, tissues, and fixed tissues. In some embodiments, the present method is used to purify a body fluid sample containing smaller cfDNA from larger gDNA. In some embodiments, the present method is used to purify small plasmid DNA from larger gDNA in a bacterial culture such as an E. coli bacterial culture. In some embodiments, the present method may be used to purify plasmids of different size. In some embodiments, the present method may be used to purify constructs of varying size such as plasmids, cosmids, fosmids, yeast artificial chromosomes, and bacterial artificial chromosomes. According to some embodiments, the nucleic acid-containing sample is a sample of extracted nucleic acid or extracted nucleic acid that has been further processed, e.g. by shearing or by way of an enzymatic reaction. In some embodiments, the nucleic acid sample is a sequencing library preparation. In some embodiments, the present method is used to purify a total RNA sample containing RNA species of different sizes. In some embodiments, the present method is used to isolate a small RNA fraction from a total RNA sample. In some embodiments, the present method is used to isolate larger rRNA or mRNA from a total RNA sample.

According to some embodiments, the nucleic acid-containing sample comprises fragmented nucleic acid, such as DNA, e.g. sheared DNA. According to other embodiments, the nucleic acid-containing sample comprises sheared genomic DNA or sheared cDNA. Thus, according to some embodiments, the nucleic acid-containing sample is a solution resulting from a size shearing procedure such as needle shearing, acoustic shearing, ultrasonic shearing, enzymatic digestion, hydrodynamic shearing, and transposase mediated fragmentation. Such a nucleic acid-containing sample comprises nucleic acid fragments of different sizes. It may be desired to obtain only DNA of a specific size or size range. Said fragmented nucleic acids can be end-repaired to provide nucleic acid fragments having blunt ends. Thus, according to some embodiments, the nucleic acid-containing sample comprises linear, blunt-ended DNA fragments of different sizes.

According to certain embodiments, the nucleic acid-containing sample is obtained after an enzymatic reaction. Exemplary enzymatic reactions that provide nucleic acid-containing samples that can be processed using the method of the disclosure includes but are not limited to polymerase chain reaction, ligation reactions, damage repair, end repair, poly-A tailing, reverse transcription, nuclease digestion, transposition, methylation, transcription, loop-mediated isothermal amplification, body labeling, and end labeling. Thus, according to some embodiments, the nucleic acid-containing sample is a solution resulting from an amplification procedure and comprises amplification products, e.g. PCR products. According to certain embodiments, the nucleic acid-containing sample is an adapter ligation sample that is obtained as a result of an adapter ligation step. In such enzymatic reactions, it may be desirous to purify the desired enzymatic reaction products from unused reactants, enzymes, reaction side products, and reaction buffers. Enzymatic reaction products can often be differentiated from reaction side products and unused reactants by size. In some embodiments, larger PCR amplification products are purified from smaller PCR primers, dNTPs, and primer dimers. In other embodiments, larger ligation products, for example gDNA-adapters, are purified from smaller pre-ligation inputs, for example unligated adapters.

In some embodiments, the enzymatic reaction is one step in a series of steps in a library preparation for sequencing. Typical library preparations for sequencing reactions include adapter ligation. According to a typical embodiment, adapters are modified or unmodified nucleic acid oligomers. Adapters can also be complexed with enzymes, other proteins or other non-nucleic acid molecules including, but not limited to, biotins. Adapters can be single stranded, double stranded, contain hairpins, and have blunt ends or one or more nucleotides overhanging at the 5′ or 3′ end. Single stranded adapters can be ligated to the 5′ or 3′ end or both 5′ and 3′ ends of a sample nucleic acid. Double stranded adapters, including those with hairpins can be ligated either by blunt end or sticky end ligation.

According to certain embodiments, hairpin adapters can be attached to sample DNA molecules utilizing polymerase-facilitated primer extension.

According to certain embodiments, the nucleic acid-containing sample is obtained during the preparation of a sequencing library, in particular during preparation of a third-generation sequencing library. According to a typical embodiment, the nucleic acid molecules in the sample have nucleic acid adapters (such as defined herein) ligated onto their 5′ or 3′ or both 3′ and 5′ ends. Thus, the sample may include unligated sample nucleic acid molecules, ligated sample nucleic acid molecules, unligated adapters, ligated adapter dimers, trimers and other combinations of adapter, plus other reagents including, but not limited to buffer species and enzymes. The method according to the present disclosure allows for size selective purification of double-stranded or single stranded nucleic acid, such as DNA molecules, that are flanked by 5′ and/or 3′ by adapters, thereby efficiently removing respective contaminants.

According to certain embodiments, the method according to the present disclosure is used after digestion of unprotected nucleic acid molecules to leave protected nucleic acid molecules. The digestions include but are not limited to Exonuclease III, Exonuclease VII, Lambda Exonuclease, Exonuclease I, Exonuclease VIII, T5 Exonuclease, T7 Exonuclease, T7 Exonuclease I.

According to certain embodiments, the method is used after completion of a library (final library molecules) to select only library molecules of a specific size or size range. In other embodiments, the method is used on nucleic acid starting materials, such that only nucleic acid molecules of a specific size or size range are input into the library preparation. In other embodiments, size selection is performed after an amplification step in library preparation. In other embodiments, size selection is performed after, but not limited to, a poly-A tailing, end-repair, nuclease digestion, damage repair, adapter ligation and/or transposition steps during library preparation.

### Short Read Elimiator Size Selection

In certain embodiments, the precipitation buffers of the present disclosure, such as those in the Short Read Eliminator kit, are capable of selecting large nucleic acid fragments (SRE size selection) for removal from a nucleic acid-containing sample to achieve a tunable cutoff of large reaction products (ranging from 50 bp (or nt) to 1000 kb). In certain embodiments, the desired size range of nucleic acids obtained using the instant method is greater than or equal to about 1000 base pairs (bp) (for double stranded nucleic acids) or 1000 nucleotides (nt) (for single stranded nucleic acids) or greater than or equal to about ≥50 bp (or nt), ≥100 bp (or nt), ≥200 bp (or nt), ≥300 bp (or nt), ≥400 bp (or nt), ≥500 bp (or nt), ≥600 bp (or nt), ≥700 bp (or nt), ≥800 bp (or nt), ≥900 bp (or nt), ≥1000 bp (or nt), ≥1500 bp (or nt), ≥2000 bp (or nt), ≥3000 bp (or nt), ≥5000 bp (or nt), ≥7000 bp (or nt), ≥8000 bp (or nt), ≥9000 bp (or nt), ≥10,000 bp (or nt), ≥20,000 bp (or nt), ≥30,000 bp (or nt), ≥40,000 bp (or nt), ≥50,000 bp (or nt), ≥60,000 bp (or nt), ≥70,000 bp (or nt), ≥80,000 bp (or nt), ≥90,000 bp (or nt), ≥100,000 bp (or nt), ≥200,000 bp (or nt), ≥500,000 bp (or nt), or ≥1,000,000 bp (or nt) (also referred to herein as “SRE size select”).

In some embodiments, SRE size select processes incorporate PVP and/or Ficoll. Suitable PVP/Ficoll molecules for use with the present method include, but are not limited to, polyvinyl pyrrolidinone (PVP), such as PVP (Mw10,000), PVP (Mw29,000), PVP (Mw40,000), PVP (Mw55,000), PVP (Mw360,000), PVP (Mw1,300,000), and/or Ficoll such as Ficoll (Mw70,000), Ficoll (Mw400,000). The MW of PVP may be from 5,000 to 5,000,000. The MW of Ficoll may be from 5,000 to 5,000,000. The concentration of PVP and/or Ficoll can be adjusted between about 0% and about 60%.

In some embodiments, a size selecting precipitation step is used. This method may be exemplified as follows: 1) a precipitation buffer, containing, but not limited to, water, buffer, salt, and PVP (Mw360,000) is added to the nucleic acid-containing sample; 2) the sample-buffer is centrifuged at 10000 g for 30 minutes at room temperature, during this step, the nucleic acid will pellet at the bottom of the tube; 3) the supernatant is removed from the tube; 4) 70% alcohol is added to the tube and centrifuged at 10000 g for 2 minutes at room temperature; 5) the 70% alcohol supernatant is removed from the tube and the nucleic acid pellet is re-suspended in elution buffer.

In certain embodiments, the SRE size select process is tuned by optimizing the amount (for example, 0.1%-40%) and/or type (of PVP and/or Ficoll in the precipitation step described herein. Combinations of PVP and Ficoll may also be used to fine tune size selection properties. These may include mixtures of PVP at different MW and/or Ficoll at different MW.

In certain embodiments, the SRE Size Select process is tuned by optimizing the NaCl concentration between, for example, 10 mM and 4 M. In certain embodiments, the SRE size select process is tuned by optimizing the precipitation binding time (2-60 minutes), temperature (4-50° C.) and or combinations thereof.

In some embodiments, the cut-off value of the SRE size select process is tuned by at least one of the following precipitation conditions: i) pH, ii) salt concentration, iii) presence or absence of chaotropic salts, iv) presence or absence of monovalent and/or divalent salts, v) alcohol type and concentration, vi) molecular crowder concentration and molecular weight, vii) species of molecular crowder, viii) precipitation time, ix) temperature during precipitation x) the presence or absence of denaturing agents xi) the presence or absence of other molecular species xii) buffer volume and xiii) combinations thereof.

In certain embodiments, molecular crowders are used to tune cut-off values of size selection. Molecular crowders change solution free energies of molecular species in a way that is highly dependent on the concentration and size of both the molecular crowder and the molecular species in question. This makes it possible to tune the solubility of nucleic acids using molecular crowders in a way that is highly dependent on the size of the nucleic acid. For example, in certain embodiments, a higher percentage of molecular crowder, such as PVP, increases excluded volume effects such that smaller molecules are increasingly brought out of solution. In another example, higher molecular weight molecular crowders, e.g., PVP 360,000, may be used to shift the molecular crowding effect to larger molecules and preferentially drive the precipitation and aggregation of larger sized nucleic acids.

### High Pass, Low Pass, and Band Pass Purifications

In certain embodiments, the present method can be used to recover a desired size range of nucleic acids that are larger than the sizing cutoff (i.e. high-pass). High-pass methods are described herein, for example.

In other embodiments, the present method can be used to recover nucleic acids that are smaller than the sizing cutoff (i.e. low pass). The low pass purification typically follows the sequence of: 1) a precipitation buffer, containing, but not limited to, water, buffer, salt, and PVP (Mw360,000) is added to the nucleic acid-containing sample; 2) the sample-buffer is centrifuged at 10000×g for 30 minutes at room temperature, during this step, nucleic acid with size above the cutoff value will pellet at the bottom of the tube and nucleic acid with size below the cutoff value will remain in the supernatant; 3) the supernatant is removed from the tube; 4) nucleic acids with size above the cutoff can now be purified from the supernatant by e.g. re-precipitating with a lower cutoff, or by some other method.

In certain embodiments, the sequential application of the present method can be used to allow a band of DNA sizes between a minimum and a maximum to be selected (i.e. band-pass). Thus, binding conditions are used such that nucleic acid molecules with size above a cut-off C₁ are pelleted, leaving those nucleic acid molecules smaller than C₁ in solution in the precipitation buffer. The precipitation buffer is then transferred to another microcentrifuge tube, for example, and additional buffer with, for example, higher PVP content is added to the original buffer. This is then centrifuged at for example 10000×g for 30 minutes. The buffer conditions are such that nucleic acid molecules with a size above a cut-off C₂ precipitate and pellet. The method then continues by washing and eluting as described elsewhere in this disclosure. The final recovered nucleic acid molecules are selected to be in a band between a minimum of C₂ and a maximum of C₁.

In certain embodiments, the purification can proceed such that both the high-pass fractions and the low-pass fractions are recovered. The high-pass method is followed as described herein. The precipitation buffer containing nucleic acids with sizes below the cutoff are removed and re-purified to give a nucleic acid stock containing nucleic acid molecules with sizes below the cutoff size.

### Sequencing Libraries

The method according to the present disclosure is particularly suitable for size selection in the context of a sequencing library, e.g., a 3rd generation sequencing library. A sequencing library which is suitable for 3rd generation sequencing, for example, can be prepared using methods known in the art. Library preparation for such long-read sequencing technologies, e.g., sequences of tens of thousands or even hundreds of thousands of base pairs, follows a similar workflow. Typically, high MW (50 kb-Mb+) DNA is isolated. Next, the DNA size selection as described in this submission may be performed to remove molecules below a cutoff length, thereby enhancing the representation of long reads in the sequencing data. This size selected DNA is then typically prepared for sequencing using various enzymatic reactions such as ligation, end repair, and labeling. During enzymatic processing, size selection as described in this submission may be performed to remove size fractions of DNA molecules (e.g. those with a tunable cut off value between 50 μL or bp and 1,000,000 μL or bp) such as primer dimers, enzymes, and adapter oligos from the library products.

In certain embodiments, the preparation of a sequencing library often involves the generation of a plurality of double-stranded, linear DNA fragments from a nucleic acid containing sample. For example, DNA, such a genomic DNA or cDNA, can be fragmented by shearing, such as sonication, hydro-shearing, ultrasound, nebulization or enzymatic fragmentation in order to provide DNA fragments that are suitable for subsequent sequencing. The length of the fragments can be chosen based on the sequencing capacity of the sequencing platform that is subsequently used for sequencing. In some embodiments of the present disclosure, larger nucleic acid fragments are selected for isolation during the preparation of a library using the method described herein for selecting larger nucleic acid molecules, e.g. those with a tunable cut off value between 50 bp to 1000 kbp.

### EXAMPLES

### Example 1

In this example, double stranded DNA is recovered above a tunable cutoff size between 1000 and 10000 bp. The cutoff size is defined as the highest Mw band that has 10% or lower recovery. This protocol is of utility for third generation long read sequencing, where it can be used instead of the time- and sample-consuming BLUE PIPPIN™ size selection instrument.

In this example, the input sample was 25 μl of a mixture containing 100 ng/μl of a 1 kbp plus ladder (Thermo Fisher Scientific Inc. part #SM1331) and 200 ng/μl of 48,502 bp linear DNA from bacteriophage lambda purchased from Thermo Fisher.

7.5 μl of 5M NaCl and 25 μl of 2×PVP (Mw=360,000) solution were added to the samples and mixed by tapping. The 2×PVP solutions were 10, 8, 6, 4, 3.5 and 3% wt/vol Polyvinylpyrrolidone (Mw=360,000) (Sigma Aldrich part #PVP360-100G) solutions. The resultant solutions were centrifuged at 8000 g and room temperature for 30 minutes. The supernatant was removed, leaving a DNA pellet. Next, 200 μl of 70% EtOH was added to the tube and centrifuged at 8000 g at room temperature for 2 minutes. The EtOH supernatant was removed, and the DNA pellet was dried by leaving the microcentrifuge tube open at room temperature for 2 minutes. The pellet was re-suspended in 25 μl of Elution Buffer (10 mM Tris-HCl, pH=9, 0.1 mM EDTA) and incubated at room temperature for 10 minutes, tapping intermittently.

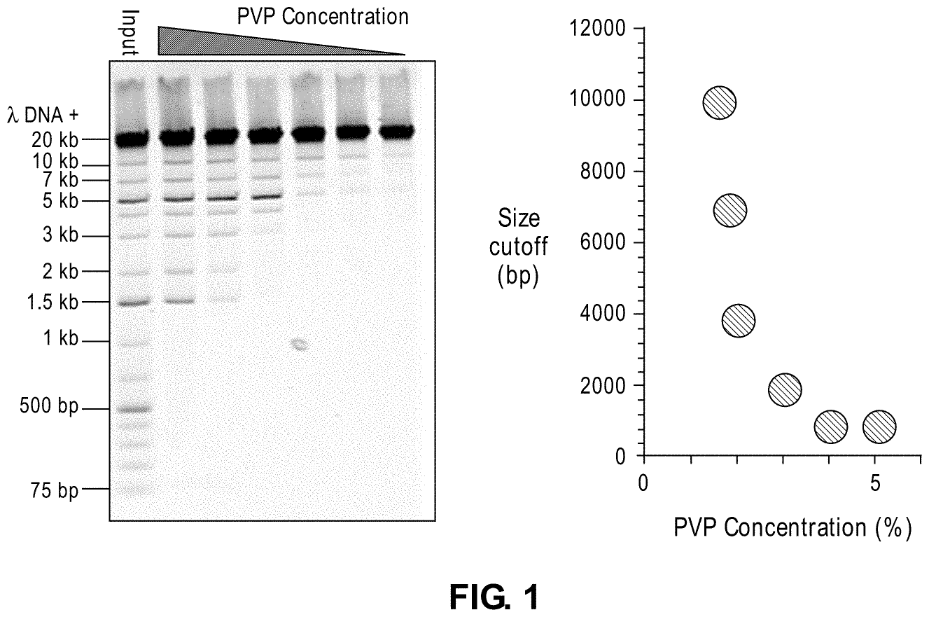

As is evident from FIG. 1, there is a significant difference in length dependent recovery as the PVP concentration in the pelleting buffer changes. The DNA cutoff (defined as the highest Mw band that has 10% or lower recovery) changes from 1000 bp to 10000 bp as PVP concentration in the buffer is decreased from 5 to 1.5%.

High size selection cutoff is essential in enhancing mean sequencing read lengths. At the same time, high recovery efficiency (>30%) of nucleic acids above the cutoff and fast processing time (<3 hours) is also desired. This combination has to date only been achievable with PVP and/or Ficoll.

### Example 2

The following exemplary protocol details size selection of HMW gDNA prior to long read sequencing library preparation for Oxford Nanopore MinION/GridION/PromethION. The input HMW DNA should have length>50 kb and QUBIT™ DNA concentration>50 ng/μL.

1. Adjust the DNA sample to a total volume of 60 μL and a QUBIT™ DNA concentration of between 50-150 ng/μL. Pipette sample into a 1.5 mL Eppendorf DNA LoBind tube. Measure the concentration using QUBIT™ dsDNA Broad Range Assay or equivalent. Dilute sample using TE buffer (pH 8) or Buffer Elution Buffer (EB).

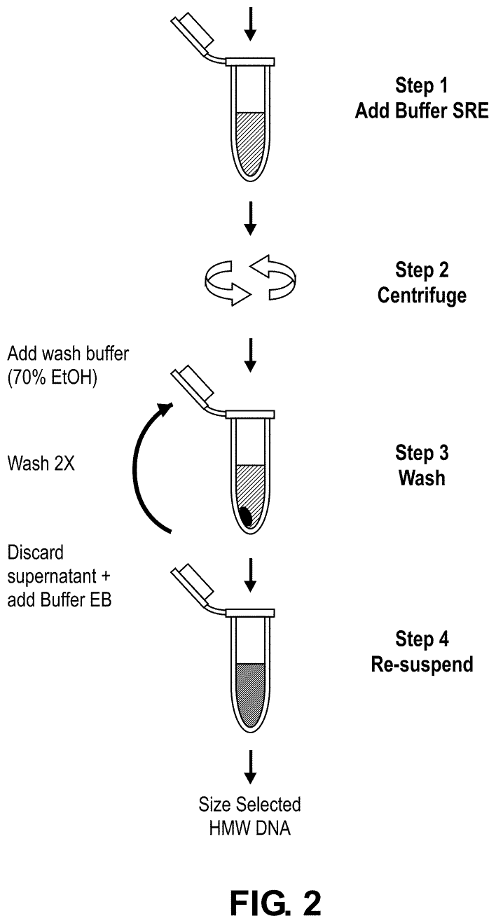

2. Add 60 μl of Buffer Short Read Eliminator (SRE) to the sample. Mix thoroughly by gently tapping the tube or by gently pipetting up and down. See Step 1 depicted in FIG. 2.

3. Load tube into centrifuge with the hinge facing toward the outside of the rotor.

4. Centrifuge at 10,000×g for 30 minutes at room temperature (RT). If using a centrifuge with temperature control (i.e. cooling function), turn this function off by setting the temperature to 29° C. See Step 2 depicted in FIG. 2.

5. Remove supernatant from tube without disturbing the DNA pellet. The DNA pellet will have formed on the bottom of the tube under the hinge region.

6. Add 200 μL of the 70% EtOH wash solution to tube and centrifuge at 10,000×g for 2 minutes at RT. Do not tap or mix after adding 70% EtOH. Place tube directly into centrifuge. See Step 3 depicted in FIG. 2.

7. Remove wash solution from tube without disturbing the DNA pellet.

8. Repeat step 6 and step 7.

9. Add 50-100 μL of Buffer Elution Buffer (EB) to the tube and incubate at 50° C. for 1 hour. Buffer volume may be adjusted to achieve desired concentration.

10. After incubation, gently tap the tube to ensure that the DNA is properly re-suspended and mixed. See Step 4 depicted in FIG. 2.

11. Analyze the recovery and purity of the DNA by NanoDrop and QUBIT™.

### Example 3

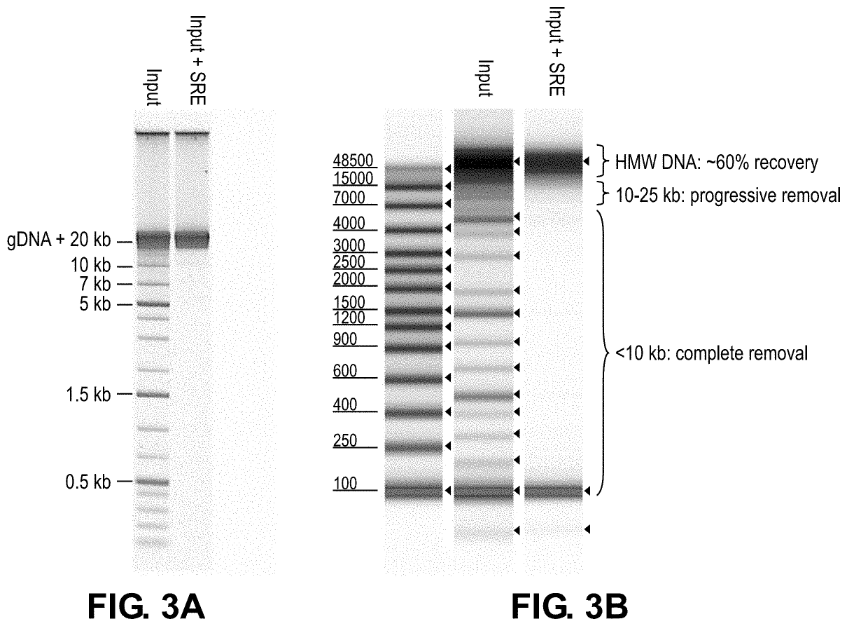

The Circulomics Short Read Eliminator (SRE) Kit can be used for rapid high-pass size selection of high molecular weight (HMW) DNA. The method can enhance mean read length by progressively removing short DNA up to 25 kb in length. See FIG. 3A, which depicts an image of a 1% agarose gel separation of HMW gDNA size-selected using the Short Read Eliminator (SRE) Kit. Sizing cutoffs were demonstrated using a spiked-in ladder (Thermo Scientific GeneRuler 1 kb Plus, #SM1334). Input was 50 gDNA extracted from GM12878 cells using the NANOBIND™ CBB Big DNA Kit+20 ng/μL ladder. Read length N50 can be increased by 10-25 kb depending on sample quality. Examples of using the kit on Oxford Nanopore MinION/GridION/PromethION sequencing platforms are provided in Examples 4 and 5. The kit uses a centrifugation procedure similar to standard ethanol precipitation techniques.

The size selection method typically uses a QUBIT™ DNA input concentration of 50-150 ng/4. It is recommended that the DNA sample concentration is determined by QUBIT™ or PicoGreen. Use of lower concentrations of DNA will generally reduce recovery efficiency. Expected yields using the SRE kit with DNA extracted using the NANOBIND™ CBB Big DNA Kit as input is shown in Table 1.

TABLE 1
Nanobind CBB Big DNA Kit—Expected Yields

| t | Required Qubit DNA | | |
|---|---|---|---|
| Sample Inpu | Input Concentration | Short DNA Removal | HMW DNA Recovery |
| HMW DNA | 50-150 ng/μL | Complete removal: <10 kb | ~60% |
| | | Progressive remove: 10 kb-25 | |
| | | kb | |

FIG. 3B depicts an image of the gel separation of the sample from FIG. 3A analyzed using an Agilent TapeStation 4200. DNA <10 kb in length was nearly completely removed as seen on agarose gel and CE analysis. DNA from 10-25 kb was progressively removed. Recovery of HMW DNA was about 60%.

### Example 4

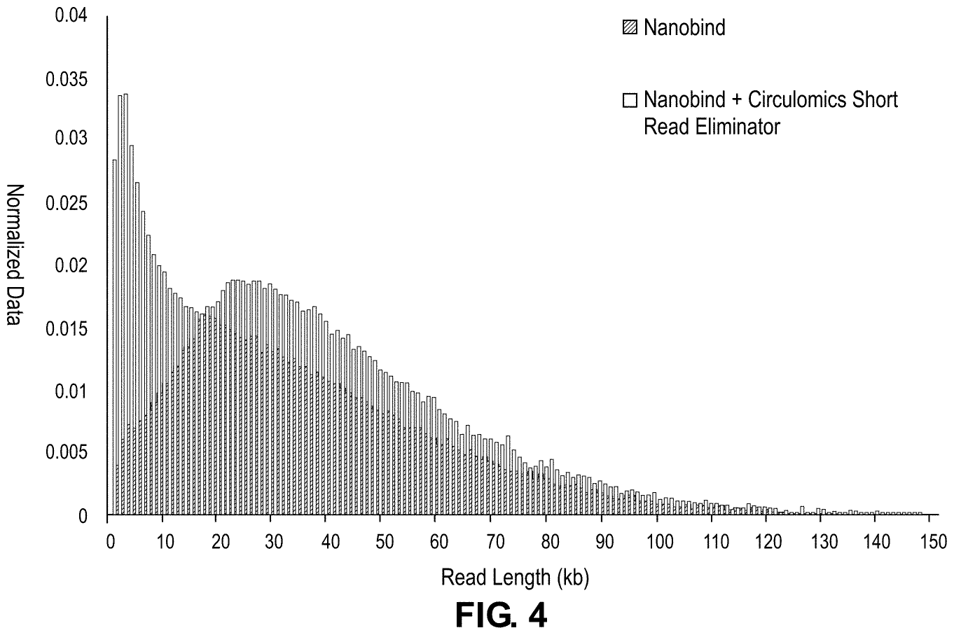

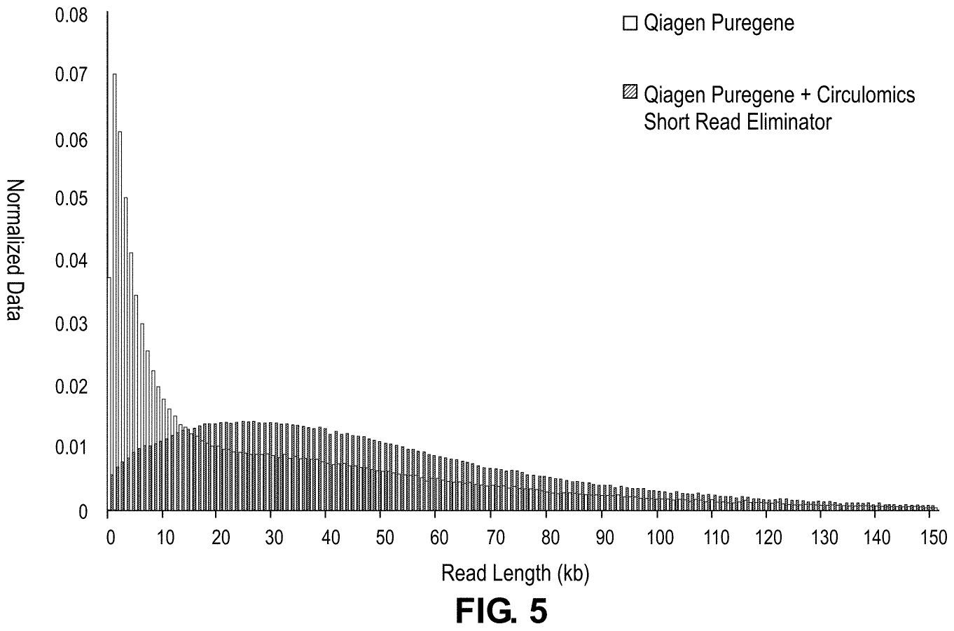

HMW DNA was extracted from GM12878 cells using the NANOBIND™ CBB Big DNA Kit, 5× needle sheared, and then sequenced on Oxford Nanopore GridION (FLO-MIN106D) using the Ligation Sequencing Kit (SQK-LSK109). Size selection of the HMW DNA using the Short Read Eliminator Kit increased N50 from 25.5 kb to 36 kb. See FIG. 4. The results of this example are further summarized in Table 2.

TABLE 2
Oxford Nanopore MinION/GridION

| | | | Read Length N50 | |
|---|---|---|---|---|
| Extraction Method | Size Selection | Shear | (bp) | Total Data (Gb) |
| Circulomics Nanobind | None | 5X Needle Shear | 25,530 | 7.6 |
| Circulomics Nanobind | Circulomics SRE | 5X Needle Shear | 36,022 | 4.9 |

### Example 5

HMW DNA was extracted from GM12878 cells using the Qiagen Puregene kit and then sequenced on Oxford Nanopore PromethION (FLO-PRO002) using the Ligation Sequencing Kit (SQK-LSK109). Size selection of the HMW DNA using the Short Read Eliminator Kit increased N50 from 17.6 kb to 40.6 kb. See FIG. 3. The results of this example are further summarized in Table 3.

TABLE 3
Oxford Nanopore PromethION

| Extraction | | | Read Length | Total |
|---|---|---|---|---|
| Method | Size Selection | Shear | N50 (bp) | Data (Gb) |
| Qiagen Puregene | None | None | 17,615 | 65.2 |
| Qiagen Puregene | Circulomics SRE | None | 40,589 | 61.2 |

### Example 6

The following protocol details the use of the Short Read Eliminator Kit after each reaction step in the SQK-LSK109 library preparation protocol for Oxford Nanopore MinION/GridION/PromethION sequencing. HMW DNA was extracted from GM12878 cells using the NANOBIND™ CBB Big DNA Kit and 5× 26G needle sheared.

The DNA sample was adjusted to a total volume of 48 μL and a QUBIT™ DNA concentration of 83 ng/μL. The sample was pipetted into a 1.5 mL Eppendorf DNA LoBind tube.

3.5 μL of NEBNext FFPE DNA Repair Buffer, 2 μL of NEBNext FFPE DNA Repair Mix, 3.5 μL of NEB Ultra II End-prep reaction buffer, 3 μL of NEB Ultra II End-prep enzyme mix were added to the sample, giving 60 μL total reaction volume, this was tapped to mix and spun down.

The reaction volume was incubated at 20° C. for 5 minutes and 65° C. for 5 minutes.

60 μL of Buffer Short Read Eliminator (SRE) was added to the sample and mixed thoroughly by gently tapping. See Step 1 depicted in FIG. 2.

The tube was loaded into a centrifuge with the hinge facing toward the outside of the rotor.

The tube was centrifuged at 10,000×g for 30 minutes at room temperature (RT). See Step 2 depicted in FIG. 2.

The supernatant was removed from tube without disturbing the DNA pellet.

200 μL of the 70% EtOH wash solution was added to tube and centrifuged at 10,000×g for 2 minutes at RT. See Step 3 depicted in FIG. 2.

The wash solution was removed from tube without disturbing the DNA pellet.

Steps [0087] and step [0088] were repeated.

61 μL of nuclease-free water was added to the tube and incubate at room temperature for 10 minutes.

After incubation, the tube was gently tapped to ensure that the DNA was properly re-suspended and mixed. See Step 4 depicted in FIG. 2.

The QUBIT™ DNA concentration was measured using 1 μL of the eluted DNA solution and found to be 25.6 ng/μL.

25 μL of Ligation Buffer (LNB) from the Oxford Nanopore Technologies SQK-LSK109 kit, 10 μL of NEBNext Quick T4 DNA Ligase, and 5 μL of Adapter Mix (AMX) from the Oxford Nanopore Technologies SQK-LSK109 kit were added to the eluted DNA solution from step [0091], giving 100 μL total ligation reaction volume.

The ligation reaction volume was incubated at room temperature for 10 minutes.

100 μL of Buffer Short Read Eliminator (SRE) was added to the sample and mixed thoroughly by gently tapping. See Step 1 depicted in FIG. 2.

The tube was loaded into a centrifuge with the hinge facing toward the outside of the rotor.

The tube was centrifuged at 10,000×g for 30 minutes at room temperature (RT). See Step 2 depicted in FIG. 2.

The supernatant was removed from tube without disturbing the DNA pellet.

250 μL of Long Fragment Buffer (LFB) from the Oxford Nanopore Technologies SQK-LSK109 kit was added to tube and centrifuged at 10,000×g for 2 minutes at RT. See Step 3 depicted in FIG. 2.

The LFB was removed from tube without disturbing the DNA pellet.

Steps [0099] and step [00100] were repeated.

20 μL of Elution Buffer (EB) from the Oxford Nanopore Technologies SQK-LSK109 kit was added to the tube and incubate at room temperature for 20 minutes.

After incubation, the tube was gently tapped to ensure that the DNA was properly re-suspended and mixed. See Step 4 depicted in FIG. 2.

The QUBIT™ DNA concentration was measured using 1 μL of the sequencing library produced in step [00103] and found to be 76.2 ng/μL.

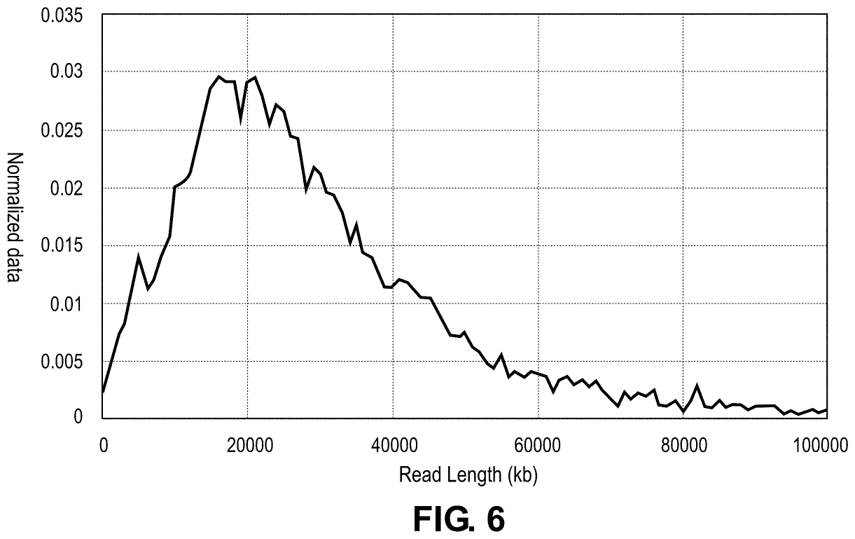

The library produced was sequenced on Oxford Nanopore MinION (FLO-MIN106D) and gave an N50 of 25 kb. FIG. 6 shows the read length distribution produced in this sequencing run. It can be seen that the reads below approximately 15 kb have been depleted by the use of the Short Read Eliminator to size select after end-prep and ligation reactions in the SQK-LSK109 library preparation protocol.

### Example 7

This example demonstrates that the methods described herein may be used to remove RNA from a nucleic acid sample comprising DNA and RNA. Total nucleic acid was extracted from 1 billion cultured E. coli cells using the NANOBIND™ CBB kit. The DNA concentration was 90 ng/ul and the RNA concentration was 335 ng/ul. 60 μL of this sample was aliquoted and the following protocol was followed:

1. 60 μL of Buffer Short Read Eliminator (SRE) to the sample. Mix thoroughly by gently tapping the tube or by gently pipetting up and down.

2. Load tube into centrifuge with the hinge facing toward the outside of the rotor.

3. Centrifuge at 10,000×g for 30 minutes at room temperature (RT). If using a centrifuge with temperature control (i.e. cooling function), turn this function off by setting the temperature to 29° C.

4. Remove supernatant from tube without disturbing the DNA pellet. The DNA pellet will have formed on the bottom of the tube under the hinge region.

5. Add 200 μL of the 70% EtOH wash solution to tube and centrifuge at 10,000×g for 2 minutes at RT. Do not tap or mix after adding 70% EtOH. Place tube directly into centrifuge.

6. Remove wash solution from tube without disturbing the nucleic acid pellet.

7. Repeat step 6 and step 7.

8. Add 50-100 μL of Buffer Elution Buffer (EB) to the tube and incubate at 50° C. for 1 hour. Buffer volume may be adjusted to achieve desired concentration.

9. After incubation, gently tap the tube to ensure that the nucleic acid is properly re-suspended and mixed.

10. Analyze the recovery and purity of the DNA by NanoDrop and QUBIT™.

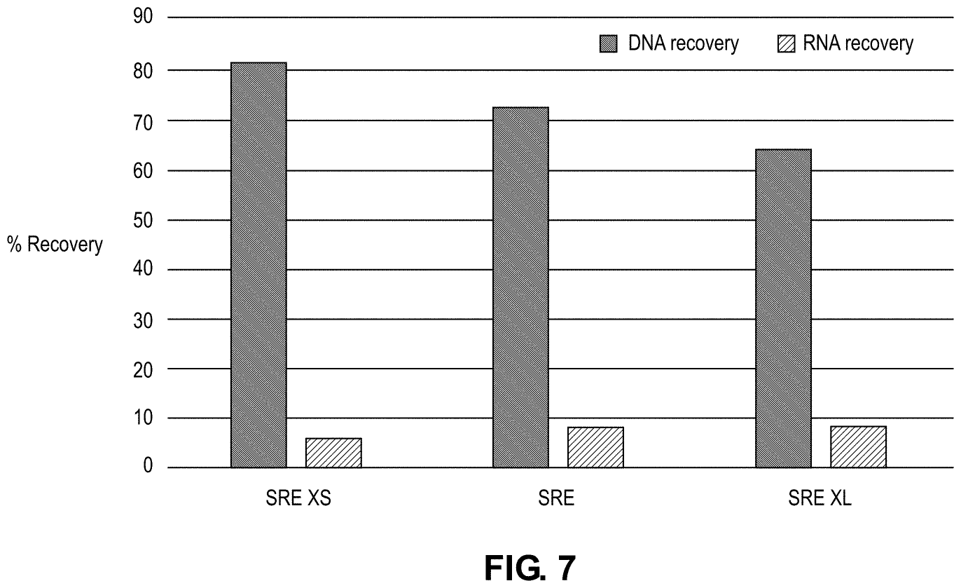

The percentage of input DNA and RNA that is recovered is shown in FIG. 7. As an example, SRE XS returns 82% of the input DNA and 6% of the input RNA as measured by the dsDNA and RNA QUBIT™ assays. Therefore, the DNA content of the sample is enriched.

### Example 8

In this example, double stranded DNA is recovered above a tunable cutoff size between 1500 and 3000 bp. The cutoff size is defined as the highest Mw band that has 10% or lower recovery. This protocol is of utility for third generation long read sequencing, where it can be used instead of the time- and sample-consuming BLUE PIPPIN™ size selection instrument.

In this example, the input sample was 40 μl of a mixture containing 20 ng/μl of a 1 kbp plus ladder (Thermo Fisher Scientific Inc. part #SM1331) and 100 ng/μl of genomic DNA extracted from 5×10⁶ GM12878 cells using NANOBIND™ CBB kit.

For Sample a) 10 μl Elution buffer from the NANOBIND™ CBB kit and 50 μl of a 40% wt/vol stock of Ficoll-400 (Sigma Aldrich part #F4375-10G) and 1.5 M NaCl solution was added to the sample. This gave a final Ficoll-400 concentration of 20% and final NaCl concentration of 0.75 M.

For Sample b) 5 μl 3 mg/ml linear acrylamide (Thermo Fisher Scientific Inc. part #AM9520) and 5 μl 5M NaCl and 50 μl of a 40% wt/vol stock of Ficoll-400 (Sigma Aldrich part #F4375-10G) solution was added to the sample. This gave a final Ficoll-400 concentration of 20% and final NaCl concentration of 1 M and final linear acrylamide concentration of 0.15 mg/ul.

For Sample c) 5 μl 3 mg/ml glycogen (Thermo Fisher Scientific Inc. part #AM9510) and 5 μl 5M NaCl and 50 μl of a 40% wt/vol stock of Ficoll-400 (Sigma Aldrich part #F4375-10G) solution was added to the sample. This gave a final Ficoll-400 concentration of 20% and final NaCl concentration of 1 M and final glycogen concentration of 0.15 mg/ul.

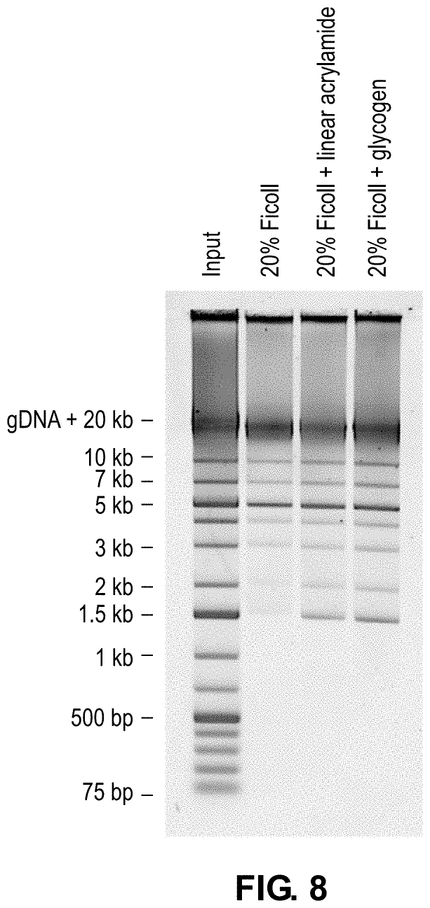

As is evident from FIG. 8, Ficoll/NaCl solutions can be used to precipitate DNA in a size dependent manner. Sample a) has a cutoff of approximately 3 kb. Sample b), which also includes linear acrylamide in the precipitation buffer has a cutoff of approximately 1.5 kb. Sample c), which also includes glycogen in the precipitation buffer has a cutoff of approximately 1.5 kb.

### Example 9

HMW DNA was extracted from leaves from Baby's breath plant using the NANOBIND™ Plant Nuclei Big DNA Kit, adapted to use a direct plant tissue lysis instead of nuclei isolation. Cleanup was performed using the Circulomics Short Read Eliminator Kit. The Extracted DNA had concentration 50 ng/μL and ratio of ultraviolet absorbance at 260 nm/280 nm=1.83 and 260 nm/230 nm=1.71. After cleanup, the concentration was 23.40 ng/μL and ratio of ultraviolet absorbance at 260 nm/280 nm=1.93 and 260 nm/230 nm=2.16.

### Example 10

HMW DNA was extracted from GM12878 cells using the NANOBIND™ CBB Big DNA Kit, 5× needle sheared, and then sequenced on PacBio Sequel II using the SMRTbell Express Template Preparation Kit 2.0 (Pacific Biosciences Part Number 100-938-900). Size selection was performed using the Circulomics Short Read Eliminator Kit after Adapter Ligation and AMPure PB beads cleanup as detailed in “Procedure and Checklist—Preparing gDNA Libraries Using the SMRTbell Express Template Preparation Kit 2.0” (Pacific Biosciences Part Number 101-693-800 Version 1 (January 2019)). The sequencing run generated 119 Gb with a subread length N50 of 28.5 kb.

1-2. (canceled)

3. A method of purifying a sample containing nucleic acids to obtain isolated nucleic acids of a desired size range, the method comprising:
a. combining a nucleic acid-containing sample with a precipitation buffer in a container to provide a precipitation mixture, wherein the precipitation buffer comprises water, a buffer, a salt, and polyvinyl pyrrolidinone (PVP) and/or Ficoll;
b. precipitating the nucleic acids in the precipitation mixture to provide a precipitated nucleic acid portion and a remaining sample portion, wherein the precipitated nucleic acid portion predominantly comprises nucleic acid molecules above a selected size cutoff value and wherein the remaining sample portion predominantly comprises nucleic acid molecules below the selected size cutoff value; and,
c. separating the precipitated nucleic acid portion from the remaining sample portion, thereby obtaining the isolated nucleic acids of the desired size range.

4. (canceled)

5. The method of claim 3, wherein the PVP comprises a molecular weight (MW) selected from the group consisting of: MW10,000, MW29,000, MW40,000, MW55,000, MW360,000, and MW1,300,000 or other molecular weights between MW5,000 and MW5,000,000.

6. The method of claim 3, wherein the Ficoll comprises a molecular weight (MW) selected from the group consisting of: MW70,000, and MW400,000 or other molecular weights between MW5,000 and MW5,000,000.

7. The method of claim 3, wherein a concentration of the PVP in the precipitation buffer is 0.1%-40% weight/volume (w/v %).

8. The method of claim 3, wherein a concentration of the Ficoll in the precipitation buffer is 0.1%-60% weight/volume (w/v %).

9. The method of claim 3, wherein the selected size cutoff value is from 50 bp-1000 kilobases (kb).

10. The method of claim 3, wherein nucleic acid molecules in the nucleic acid-containing sample comprise a concentration range of between about 1-2,000 ng/μL.

11. The method of claim 3, comprising tuning at least one condition of the precipitation buffer to determine the selected size cutoff value, wherein the condition is selected from the group consisting of: PVP concentration, PVP molecular weight, Ficoll concentration, Ficoll MW, presence or absence of chaotropic salts, presence or absence of monovalent and/or divalent salts, salt concentration and type, alcohol type and concentration, presence or absence of polyamines, presence or absence of denaturing agents, presence or absence of other additive molecules, pH, precipitation/binding time, precipitation/binding temperature, precipitation/binding volume, centrifugation time, centrifugation temperature, and combinations thereof.

12. The method of claim 3, wherein step b) comprises centrifuging the precipitation mixture.

13. The method of claim 3, wherein step b) comprises centrifuging the precipitation mixture at 10000 g for 30 minutes at room temperature (RT).

14. The method of claim 3, wherein the salt comprises one or more of: guanidinium chloride, guanidinium hydrochloride, lithium perchlorate, guanidinium thiocyanate, guanidinium isothiocyanate, sodium chloride, potassium chloride, lithium chloride, magnesium chloride, calcium chloride, sodium acetate, potassium acetate, and sodium iodide.

15. The method of claim 3, wherein the remaining sample portion comprises supernatant and wherein step c) comprises removing the supernatant from the container.

16. The method of claim 3, further comprising:
d. washing the nucleic acid pellet one or more times with an alcohol solution to produce a washed nucleic acid pellet; and,
e. resuspending the washed nucleic acid pellet in a resuspension buffer to produce resuspended nucleic acids.

17. The method of claim 16, wherein the resuspension buffer comprises a TE buffer or a low EDTA TE buffer.

18. The method of claim 3, further comprising prior to step a) combining the nucleic acid-containing sample with a binding buffer to provide a binding mixture; contacting the binding mixture with a nanomembrane, wherein the nanomembrane binds nucleic acids in the binding mixture to produce bound nucleic acids; and separating the bound nucleic acids from remaining components of the binding mixture.

19. The method of claim 3, further comprising contacting the nucleic acids with a nanomembrane in the precipitation mixture, wherein the nanomembrane binds nucleic acids in the precipitation mixture to produce bound nucleic acids during steps a), b), and/or c), d), and/or e).

20. The method of claim 16, further comprising during or after step e) contacting the resuspended nucleic acids with a nanomembrane, wherein the nanomembrane binds nucleic acids in the resuspension buffer to produce bound nucleic acids; and separating the bound nucleic acids from remaining components of the resuspension buffer.

21. The method of claim 3, further comprising sequencing the isolated nucleic acids of the desired size range after step c) to produce sequencing reads.

22. The method of claim 21, wherein an N50 of the sequencing reads obtained after performing at least steps a)-c) is increased relative to an N50 of sequencing reads obtained in the absence of performing steps a)-c).

23. A composition, comprising nucleic acids and a precipitation buffer, wherein the precipitation buffer comprises water, a buffer, a salt, and polyvinyl pyrrolidinone (PVP) and/or Ficoll, wherein a portion of the nucleic acids are present in a nucleic acid pellet that predominantly comprises nucleic acid molecules above a selected size cutoff value and wherein a remaining portion of the nucleic acids are present in a supernatant that predominantly comprises nucleic acid molecules below the selected size cutoff value.

24. The composition of claim 23, wherein the PVP comprises a molecular weight (MW) selected from the group consisting of: MW10,000, MW29,000, MW40,000, MW55,000, MW360,000, and MW1,300,000 or other molecular weights between MW5,000 and MW5,000,000.

25. The composition of claim 23, wherein the Ficoll comprises a molecular weight (MW) selected from the group consisting of: MW70,000, and MW400,000 or other molecular weights between MW5,000 and MW5,000,000.

26. The composition of claim 23, wherein a concentration of the PVP in the precipitation buffer is 0.1%-40% weight/volume (w/v %).

27. The composition of claim 23, wherein a concentration of the Ficoll in the precipitation buffer is 0.1%-60% weight/volume (w/v %).

28. The composition of claim 23, wherein the selected size cutoff value is from 50 bp-1000 kilobases (kb).

29. A kit for purifying a sample containing nucleic acids to obtain isolated nucleic acids of a desired size range, comprising a buffer, a salt, and polyvinylpyrrolidone (PVP) and/or Ficoll disposed in one or more containers.

30. The kit of claim 29, comprising a single container that comprises a precipitation buffer that comprises water, the buffer, the salt, and the PVP and/or Ficoll.

31. The kit of claim 29, further comprising a nanomembrane.

32. The kit of claim 29, further comprising one or more wash buffers and/or one or more elution buffers.

33. The kit of claim 29, further comprising one or more sequencing reagents.
